(19) United States
(12) Patent Application Publication
(10) Pub. No.: US 2019/0225607 A1
(43) Pub. Date: Jul. 25, 2019

(54) Deuterated Venetoclax

(71) Applicant: CONCERT Pharmaceuticals, Inc., Lexington, MA (US)

(72) Inventor: Roger Tung, Ph.D., Lexington, MA (US)

(21) Appl. No.: 16/315,550

(22) PCT Filed: Jun. 30, 2017
(86) PCT No.: PCT/US17/40280

§ 371 Date: Jan. 4, 2019

Related U.S. Application Data
(60) Provisional application No. 62/358,999, filed on Jul. 6, 2016.

(51) Int. Cl. C07D 471/04; C07B 59/00; A61K 45/06; A61K 31/635; A61P 35/00

(52) U.S. Cl. CPC C07D 471/04; C07B 59/002; C07B 2200/05; A61K 31/635; A61P 35/00; A61K 45/06

(57) ABSTRACT

This invention relates to deuterated forms of venetoclax, and pharmaceutically acceptable salts thereof. Certain aspects of this invention also provide pharmaceutical compositions comprising a compound of the present invention and a pharmaceutically acceptable carrier. Certain aspects of the present invention also provide the use of such compounds and compositions in methods of treating diseases and conditions that are beneficially treated by administering an inhibitor of the anti-apoptotic activity of the B-cell lymphoma 2 (Bcl-2) protein.

### RELATED APPLICATION

This application claims the benefit of U.S. Provisional Application No. 62/358,999, filed on Jul. 6, 2016. The entire teachings of this application are incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds, deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

### SUMMARY OF THE INVENTION

This invention relates to deuterated forms of N-(phenylsulfonyl)benzamide compounds, and pharmaceutically acceptable salts thereof. Certain aspects of the present invention provide a compound of Formula I:

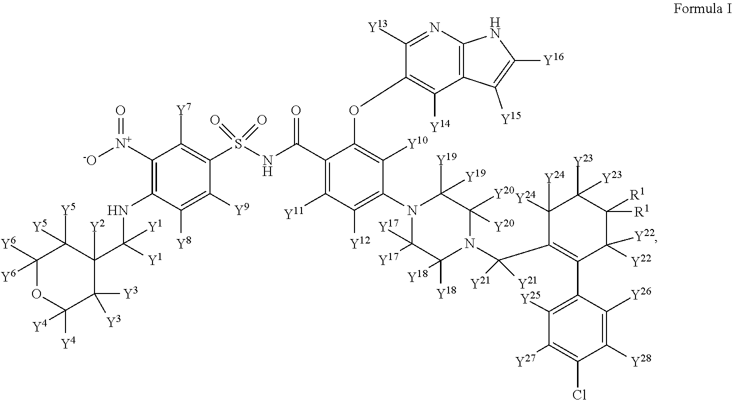

or a pharmaceutically acceptable salt thereof, wherein each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium; each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and each R¹ is independently selected from —CH₃, —CH₂D, —CHD₂ and —CD₃; wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises deuterium; provided that when each of Y¹⁷, Y¹⁸, Y¹⁹, and Y²⁰ , is deuterium, then at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y²¹, Y²², Y²³, Y²⁴, Y²⁵, Y²⁶, Y²⁷, Y²⁸, and R¹ comprises deuterium.

Certain aspects of this invention also provide compositions comprising a compound of this invention, including pharmaceutical compositions comprising a compound of this invention and a pharmaceutically acceptable carrier. Certain aspects of the present invention also provide the use of such compounds and compositions in methods of treating diseases and conditions that are beneficially treated by administering an inhibitor of the B-cell lymphoma 2 (Bcl-2) protein. Some exemplary embodiments include a method of treating a disease or condition selected from chronic lymphocytic leukemia, acute myeloid leukemia, diffuse large B-cell leukemia, follicular lymphoma, mantle cell lymphoma, non-Hodgkin's lymphoma, multiple myeloma, Waldenstrom's macroglobulinemia, and systemic lupus erythematosus, the method comprising administering to a subject in need thereof a pharmaceutically acceptable composition of the present invention.

### DETAILED DESCRIPTION OF THE INVENTION

Venetoclax is marketed under the brand name Venclexta, and is also known as ABT-199, GDC-0199, RG-7601, and chemically as 4-(4-[[2-(4-chlorophenyl)-4,4-dimethylcyclohex-1-en-1-yl]methyl]piperazin-1-yl)-N-([3-nitro-4-[(tetrahydro-2H-pyran-4-ylmethyl)amino]phenyl]sulfonyl)-2-(1H-pyrrolo[2,3-b]pyridin-5-yloxy) benzamide. Venetoclax modulates the activity of the B-cell lymphoma 2 (Bcl-2) protein and is thought to be specifically a Bcl-2 homology domain 3 (BH3) mimetic that inhibits the anti-apoptotic activity of Bcl-2.

Venetoclax was approved by the United States Food and Drug Administration (FDA) in April 2016 for treatment of patients with chronic lymphocytic leukemia (CLL) who have a 17p deletion (deletion located on the short arm of chromosome 17) and who have received at least one prior therapy. Venetoclax was also approved in the European Union in 2016 as a second-line treatment for CLL with a 17p deletion or a TP53 mutation, and as a third-line treatment for CLL without the deletion or mutation. Venetoclax is in Phase III clinical trials for relapsed/refractory multiple myeloma, acute myeloid leukemia, diffuse large B-cell leukemia, and follicular lymphoma. Venetoclax is also in Phase II clinical trials for the treatment of myelodysplasia, mantle cell lymphoma, non-Hodgkin's lymphoma, and Waldenstrom's macroglobulinemia. Early clinical development (Phase I) has been initiated for treatment of systemic lupus erythematosus and amyloidosis.

Patients on venetoclax have experienced the following undesirable side effects: neutropenia, diarrhea, nausea, infection, anemia, thrombocytopenia, and fatigue, among others.

Despite the beneficial activities of venetoclax, there is a continuing need for new compounds to treat the aforementioned diseases and conditions.

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “subject” includes humans and non-human mammals. Non-limiting examples of non-human mammals include mice, rats, guinea pigs, rabbits, dogs, cats, monkeys, apes, pigs, cows, sheep, horses, etc.

It will be recognized that some variation of natural isotopic abundance occurs in a synthesized compound depending upon the origin of chemical materials used in the synthesis. Thus, a preparation of venetoclax will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. In some embodiments, when a position is designated specifically as “H” or “hydrogen”, the position has at least 80%, at least 90%, at least 95%, at least 96%, at least 97%, at least 98%, or at least 99% hydrogen. In some embodiments, when a position is designated specifically as “H” or “hydrogen”, the position incorporates ≤20% deuterium, ≤10% deuterium, ≤5% deuterium, ≤4% deuterium, ≤3% deuterium, ≤2% deuterium, or ≤1% deuterium. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, in a compound of this invention, each designated deuterium atom has deuterium incorporation of at least 52.5%. In some embodiments, in a compound of this invention, each designated deuterium atom has deuterium incorporation of at least 60%. In some embodiments, in a compound of this invention, each designated deuterium atom has deuterium incorporation of at least 67.5%. In some embodiments, in a compound of this invention, each designated deuterium atom has deuterium incorporation of at least 75%. In some embodiments, in a compound of this invention, each designated deuterium atom has deuterium incorporation of at least 82.5%. In some embodiments, in a compound of this invention, each designated deuterium atom has deuterium incorporation of at least 90%. In some embodiments, in a compound of this invention, each designated deuterium atom has deuterium incorporation of at least 95%. In some embodiments, in a compound of this invention, each designated deuterium atom has deuterium incorporation of at least 97.5%. In some embodiments, in a compound of this invention, each designated deuterium atom has deuterium incorporation of at least 99%. In some embodiments, in a compound of this invention, each designated deuterium atom has deuterium incorporation of at least 99.5%.

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound.

The invention also provides salts of the compounds of the invention.

A salt of a compound of this invention is formed between an acid and a basic group of the compound, such as an amino functional group, or a base and an acidic group of the compound, such as a carboxyl functional group. According to one embodiment, the compound is a pharmaceutically acceptable acid addition salt. In one embodiment, the acid addition salt may be a deuterated acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In one embodiment, the acids commonly employed to form pharmaceutically acceptable salts include the above-listed inorganic acids, wherein at least one hydrogen is replaced with deuterium.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. “Stereoisomer” refers to both enantiomers and diastereomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “US” refers to the United States of America.

“Substituted with deuterium” refers to the replacement of one or more hydrogen atoms with a corresponding number of deuterium atoms.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

Certain aspects of the present invention provide a compound of Formula I:

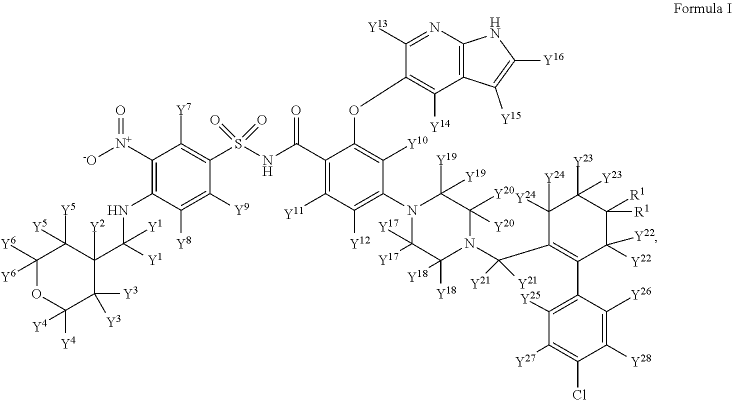

or a pharmaceutically acceptable salt thereof, wherein each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium; each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and each R¹ is independently selected from —CH₃, —CH₂D, —CHD₂ and —CD₃; wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises deuterium; provided that when each of Y¹⁷, Y¹⁸, Y¹⁹, and Y²⁰ is deuterium, then at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y²¹, Y²², Y²³, Y²⁴, Y²⁵, Y²⁶, Y²⁷, Y²⁸, and R¹ comprises deuterium.

In some embodiments, each R¹ is the same, each Y¹ is the same, each Y³ is the same, each Y⁴ is the same, each Y⁵ is the same, each Y⁶ is the same, each Y¹⁷ is the same, each Y¹⁸ is the same, each Y¹⁹ is the same, each Y²⁰ is the same, each Y²¹ is the same, each Y²² is the same, each Y²³ is the same, and each Y²⁴ is the same.

In some embodiments, R¹ is —CH₃ or —CD₃. In some embodiments, R¹ is —CD₃. In some embodiments, R¹ is —CH₃.

In some embodiments, Y²⁵ and Y²⁶ are the same, and Y²⁷ and Y²⁸ are the same. In some embodiments, Y²⁵ and Y²⁶ are each hydrogen. In some embodiments, Y²⁷ and Y²⁸ are each hydrogen. In some embodiments, Y²⁵ and Y²⁶ are each deuterium. In some embodiments, Y²⁷ and Y²⁸ are each deuterium. In some embodiments, each of Y²⁵, Y²⁶, Y²⁷ and Y²⁸ is hydrogen. In some embodiments, each of Y²⁵, Y²⁶, Y²⁷ and Y²⁸ is deuterium. In some embodiments, Y²⁵ and Y²⁶ are each hydrogen and Y²⁷ and Y²⁸ are each deuterium. In some embodiments, Y²⁵ and Y²⁶ are each deuterium and Y²⁷ and Y²⁸ are each hydrogen.

In some embodiments, Y² and each Y¹, Y³, Y⁴, Y⁵, and Y⁶ are the same. In some embodiments, Y² and each Y¹, Y³, Y⁴, Y⁵, and Y⁶ are deuterium. In some embodiments, Y² and each Y¹, Y³, Y⁴, Y⁵, and Y⁶ are hydrogen. In some embodiments, each Y¹, Y⁴, and Y⁶ is deuterium, and Y² and each Y³ and Y⁵ are hydrogen. In some embodiments, each Y¹, Y⁴, and Y⁶ is hydrogen, and Y² and each Y³ and Y⁵ are deuterium. In some embodiments, each Y⁴ and Y⁶ is deuterium and Y² and each Y¹, Y³, and Y⁵ are hydrogen. In some embodiments, each Y⁴ and Y⁶ is hydrogen and Y² and each Y¹, Y³, and Y⁵ are deuterium. In some embodiments, each Y¹, Y³, Y⁴, Y⁵, and Y⁶ is deuterium, and Y² is hydrogen. In some embodiments, each Y¹, Y³, Y⁴, Y⁵, and Y⁶ is hydrogen, and Y² is deuterium.

In some embodiments, Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ are the same. In some embodiments, each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is hydrogen. In some embodiments, each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is deuterium. In some embodiments, each of Y¹³, Y¹⁴, Y¹⁵, and Y¹⁶ is deuterium. In some embodiments, each of Y¹³ and Y¹⁶ is deuterium, and each of Y¹⁴ and Y¹⁵ is hydrogen.

In some embodiments, each Y¹⁷ and Y¹⁹ is deuterium, and each Y¹⁸ and Y²⁰ is hydrogen. In some embodiments, each Y¹⁷ and Y¹⁹ is hydrogen, and each Y¹⁸ and Y²⁰ is deuterium. In some embodiments, when each Y¹⁷, Y¹⁸, Y¹⁹, and Y²⁰ is deuterium, then at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y²¹, Y²², Y²³, Y²⁴, Y²⁵, Y²⁶, Y²⁷, Y²⁷, Y²⁸, and R¹ comprises deuterium.

In some embodiments, each Y²¹, Y²², Y²³, and Y²⁴ is the same. In some embodiments, each Y²¹, Y²², Y²³, and Y²⁴ is deuterium. In some embodiments, each Y²¹ is hydrogen, and each Y²², Y²³, and Y²⁴ is deuterium. In some embodiments, each Y²¹, Y²², and Y²³ is hydrogen, and each Y²⁴ is deuterium. In some embodiments, each Y²¹, Y²³, and Y²⁴ is hydrogen, and each Y²² is deuterium. In some embodiments, each Y²¹, Y²², and Y²⁴ is hydrogen, and each Y²³ is deuterium. In some embodiments, each Y²² and Y²⁴ is deuterium, and each Y²¹ and Y²² is hydrogen.

In some embodiments of a compound of this invention, at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises hydrogen.

In some embodiments, each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is hydrogen; Y² and each Y¹, Y³, Y⁴, Y⁵, and Y⁶ are the same; each Y¹⁷ and Y¹⁹ is the same; each Y¹⁸ and Y²⁰ is the same; each Y²¹, Y²², Y²³, and Y²⁴ is the same; each R¹ is the same; and the compound is selected from any one of the compounds set forth in Table 1 (below):

TABLE 1

| Compound | Y¹⁻⁶ | Y¹⁷/Y¹⁹ | Y¹⁸/Y²⁰ | Y²¹⁻²⁴ | R¹ |
|---|---|---|---|---|---|
| 101 | D | H | H | H | CH₃ |
| 102 | D | D | H | H | CH₃ |
| 103 | D | H | D | H | CH₃ |
| 104 | D | H | H | D | CH₃ |
| 105 | D | D | D | H | CH₃ |
| 106 | D | H | D | D | CH₃ |
| 107 | D | D | H | D | CH₃ |
| 108 | D | D | D | D | CH₃ |
| 109 | D | H | H | H | CD₃ |
| 110 | D | D | H | H | CD₃ |
| 111 | D | H | D | H | CD₃ |
| 112 | D | H | H | D | CD₃ |
| 113 | D | D | D | H | CD₃ |
| 114 | D | H | D | D | CD₃ |
| 115 | D | D | H | D | CD₃ |
| 116 | D | D | D | D | CD₃ |
| 117 | H | D | H | H | CH₃ |
| 118 | H | H | D | H | CH₃ |
| 119 | H | H | H | D | CH₃ |
| 120 | H | D | D | H | CH₃ |
| 121 | H | H | D | D | CH₃ |
| 122 | H | D | H | D | CH₃ |
| 123 | H | D | D | D | CH₃ |
| 124 | H | H | H | H | CD₃ |
| 125 | H | D | H | H | CD₃ |
| 126 | H | H | D | H | CD₃ |
| 127 | H | H | H | D | CD₃ |
| 128 | H | D | D | H | CD₃ |
| 129 | H | H | D | D | CD₃ |
| 130 | H | D | H | D | CD₃ |
| 131 | H | D | D | D | CD₃, |

or a pharmaceutically acceptable salt of any of the foregoing.

In some embodiments, each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is hydrogen; each Y¹, Y⁴, and Y⁶ is deuterium; Y² and each Y³, and Y⁵ are hydrogen; each Y¹⁷ and Y¹⁹ is the same; each Y¹⁸ and Y²⁰ is the same; each Y²¹, Y²², Y²³, and Y²⁴ are the same; each R¹ is the same; and the compound is selected from any one of the compounds set forth in Table 2 below:

TABLE 2

| Compound | Y¹⁷/Y¹⁹ | Y¹⁸/Y²⁰ | Y²¹⁻²⁴ | R¹ |
|---|---|---|---|---|
| 201 | H | H | H | CH₃ |
| 202 | D | H | H | CH₃ |
| 203 | H | D | H | CH₃ |
| 204 | H | H | D | CH₃ |
| 205 | D | D | H | CH₃ |
| 206 | H | D | D | CH₃ |
| 207 | D | H | D | CH₃ |
| 208 | D | D | D | CH₃ |
| 209 | H | H | H | CD₃ |
| 210 | D | H | H | CD₃ |
| 211 | H | D | H | CD₃ |
| 212 | H | H | D | CD₃ |
| 213 | D | D | H | CD₃ |
| 214 | H | D | D | CD₃ |
| 215 | D | H | D | CD₃ |
| 216 | D | D | D | CD₃, |

or a pharmaceutically acceptable salt of any of the foregoing.

In another set of embodiments, any atom not designated as deuterium in any of the embodiments set forth above is present at its natural isotopic abundance.

In some embodiments of a compound of this invention, when one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, and Y²⁸ is deuterium, the level of deuterium incorporation at each of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, and Y²⁸ which is designated as deuterium is at least 52.5%, at least 75%, at least 82.5%, at least 90%, at least 95%, at least 97%, or at least 99%.

In some embodiments, when R¹ is CD₃, the level of deuterium incorporation at each R¹ is at least 52.5%, at least 75%, at least 82.5%, at least 90%, at least 95%, at least 97%, or at least 99%.

In some embodiments of a compound of this invention, at least one Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises hydrogen.

The present invention also provides deuterated intermediates useful, e.g., in the preparation of the compounds of Formula I, and as provided in the Exemplary Schemes.

The synthesis of compounds of Formula I may be readily achieved by synthetic chemists of ordinary skill by reference to the Exemplary Synthesis and Examples disclosed herein. Relevant procedures analogous to those of use for the preparation of compounds of Formula I and intermediates thereof are disclosed, for instance in CN104370905.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I is depicted in Scheme 1, below.

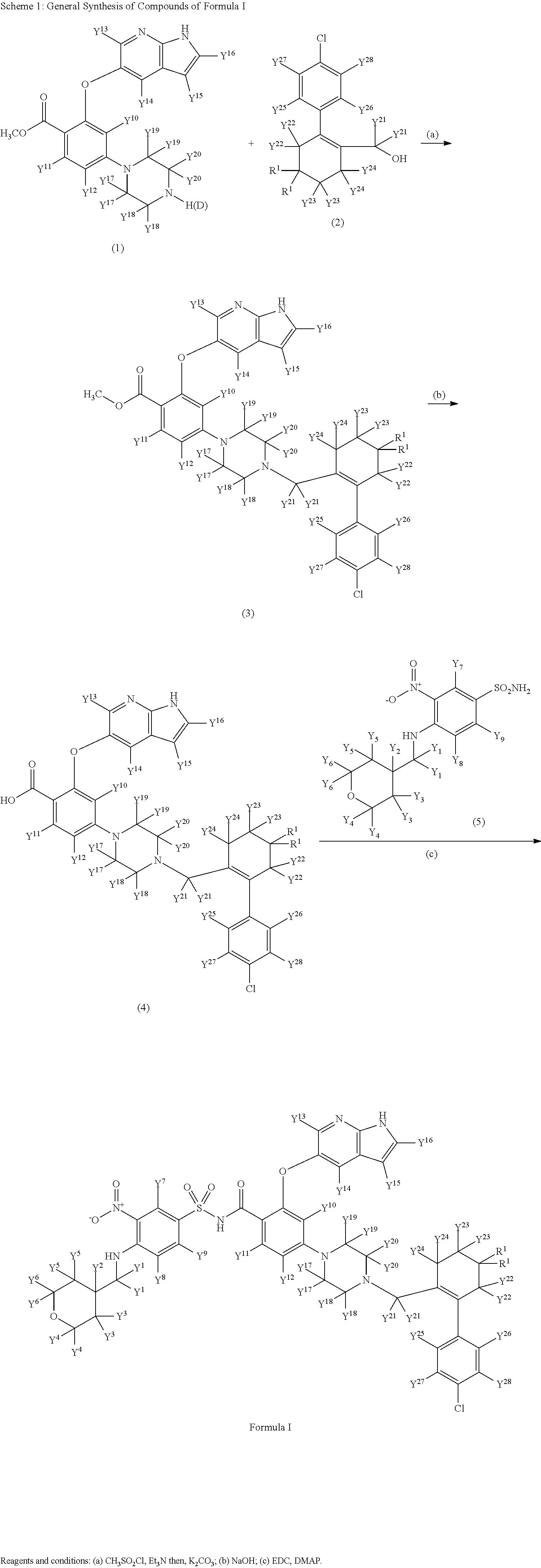

In a manner analogous to a procedure described in CN104370905, in situ treatment of appropriately deuterated alcohol intermediate (2) with methanesulfonyl chloride in the presence of a base such as triethylamine generates a mesylate, which is then alkylated with appropriately deuterated piperazine intermediate (1) using potassium carbonate as a base, to furnish appropriately deuterated alkylpiperazine intermediate (3). Base hydrolysis of the ester moiety in (3) with sodium hydroxide affords appropriately deuterated carboxylic acid intermediate (4), which is subsequently coupled with appropriately deuterated sulfonamide intermediate (5) using N-(3-dimethylaminopropyl)-N′-ethylcarbodiimide hydrochloride (EDC) in the presence of catalytic amounts of 4-(dimethylamino)pyridine (DMAP), or using standard methods known in the art, to produce appropriately deuterated acyl sulfonamide compounds of Formula I.

Using commercially available reagents and deuterated reagents that can be readily prepared by known methods, compounds of Formula I can be prepared with greater than 90%, greater than 95%, greater than 97%, or greater than 99% deuterium incorporation at each position designated as D (see below for details).

Appropriately deuterated intermediate (1), for use in the preparation of compounds of Formula I according to Scheme 1, may be prepared from corresponding deuterated reagents exemplified in Scheme 2.

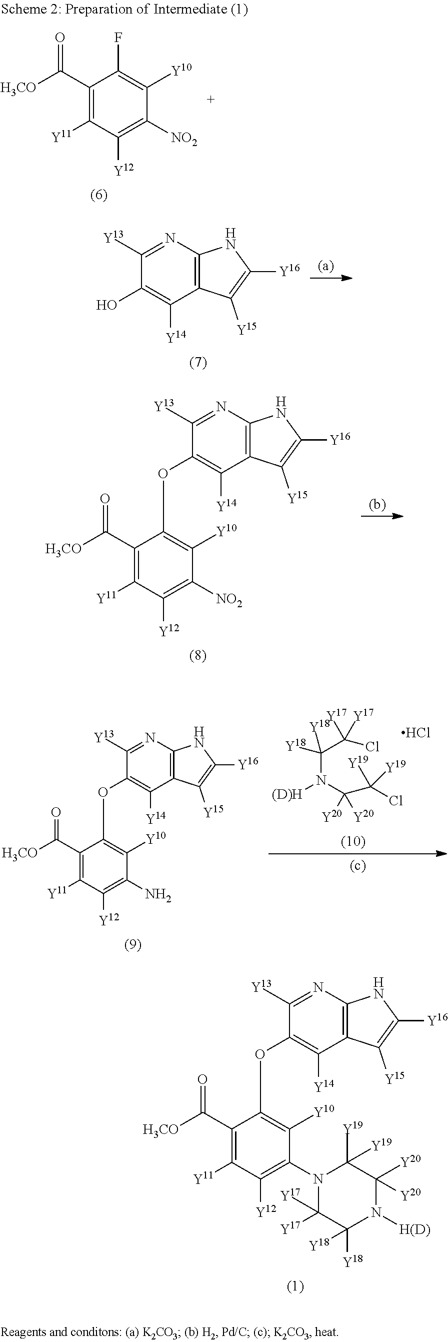

By analogy to a procedure described in CN104370905, displacement of fluoride in appropriately deuterated benzoate intermediate (6) with appropriately deuterated hydroxyazaindole (7) in the presence of a base such as potassium carbonate affords appropriately deuterated intermediate azaindolylbenzoate intermediate (8). Subsequent hydrogenation of nitro moiety in (8) in the presence of palladium on carbon furnishes appropriately deuterated aniline intermediate (9). Finally, alkylation of (9) with appropriately deuterated bis-chloroethylamine intermediate (10) using potassium carbonate as base and at elevated temperature produces appropriately deuterated piperazine intermediate (1).

The following intermediate (10) are commercially available: Bis(2-chloroethyl)-d₈-amine HCl (98 atom %D) (10a), Bis(2-chloroethyl)amine-d₄ Hydrochloride (98 atom %D) (10b), Bis(2-chloroethyl)-1,1,2,2-d₄-amine HCl (98 atom %D) (10c). Ethan-1,1-d₂-amine, 2-chloro-N-(2-chloroethyl-2,2-d₂)-, hydrochloride, intermediate (10d), may be prepared according to a procedure described by Springer, J. et al., Journal of Labelled Compounds and Radiopharmaceuticals, 50(2), 115-122; 2007.

Use of appropriately deuterated reagents allows deuterium incorporation at the Y¹⁰, Y¹¹, Y¹², Y¹³, Y¹⁴, Y¹⁵, Y¹⁶, Y¹⁷, Y¹⁸, Y¹⁹, Y²⁰ positions of a compound of Formula I or any appropriate intermediate herein, e.g., about 90%, about 95%, about 97%, or about 99% deuterium incorporation at any Y¹⁰, Y¹¹, Y¹², Y¹³, Y¹⁴, Y¹⁵, Y¹⁶, Y¹⁷, Y¹⁸, Y¹⁹, and/or Y²⁰.

Appropriately deuterated intermediate (6), for use in the preparation of intermediate (1) according to Scheme 2, which is used to prepare compounds of Formula I according to Scheme 1, may be prepared from corresponding deuterated reagents exemplified in Scheme 3.

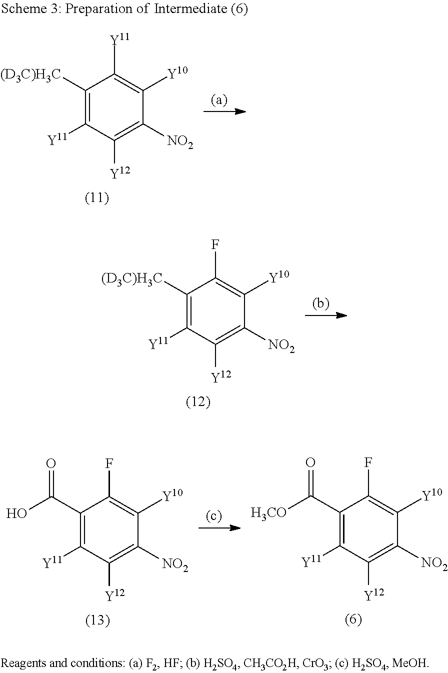

In a manner analogous to a procedure described in WO 2001079143, direct fluorination of appropriately deuterated nitrotoluene intermediate (11) using fluorine gas in the presence of hydrofluoric acid furnishes appropriately deuterated fluoronitrotoluene intermediate (12). Oxidation of methyl moiety in (12) by analogy to a procedure described in WO2001009107, using chromium oxide in the presence of sulfuric acid and acetic acid affords appropriately deuterated benzoic acid intermediate (13), which is subsequently esterified by analogy to a procedure described in WO 2013068467 using sulfuric acid and methanol or using standard methods known in the art, to produce appropriately deuterated benzoate intermediate (6).

Intermediate (11) is commercially available: 4-Nitrotoluene-2,3,5,6-d₄ (99 atom %D) (11a).

Use of appropriately deuterated reagents allows deuterium incorporation at the Y¹⁰, Y¹¹, Y¹² positions of a compound of Formula I or any appropriate intermediate herein, e.g., about 90%, about 95%, about 97%, or about 99% deuterium incorporation at any Y¹⁰, Y¹¹, and/or Y¹².

Appropriately deuterated intermediate (7), for use in the preparation of intermediate (1) according to Scheme 2, which is used to prepare compounds of Formula I according to Scheme 1, may be prepared from corresponding deuterated reagents as exemplified in Scheme 4.

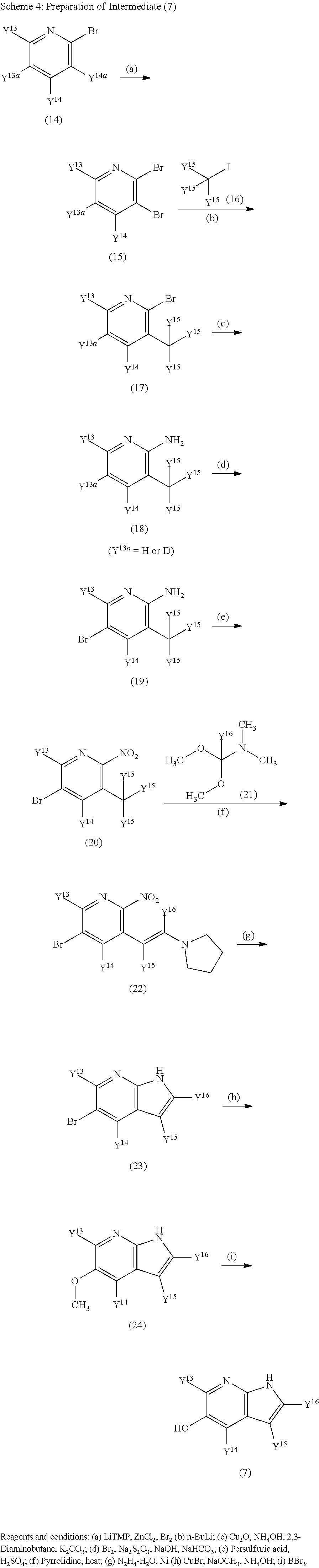

By analogy to a procedure described by Menzel, K. et al., Journal of Organic Chemistry, 71(5), 2188-2191; 2006, appropriately deuterated bromopyridine intermediate (14) is transiently ortho-lithiated using a sterically hindered base such as lithium 2,2,6,6-tetramethylpiperidide (LiTMP) and then transmetalated with zinc chloride to furnish appropriately deuterated pyridylzinc species which is subsequently treated with bromine to afford appropriately deuterated dibromide intermediate (15). Selective bromine-lithium exchange followed by quenching with appropriately deuterated iodomethane intermediate (16), by analogy to a procedure described by Nagaki, A. et al., Australian Journal of Chemistry, 66(2), 199-207; 2013, affords correspondingly and appropriately deuterated methylpyridine intermediate (17). Copper catalyzed amination of bromide (17) using aqueous ammonia, furnishes appropriately deuterated aminopicoline intermediate (18) in a manner analogous to a procedure described by Elmkaddem, M. et al., Chemical Communications (Cambridge, United Kingdom), 46(6), 925-927; 2010. Treatment of (18) with bromine affords appropriately deuterated bromide intermediate (19) by analogy to a procedure described in WO 2004013139, and oxidation of amino moiety in (19) with persulfuric acid (Caro's acid) furnishes appropriately deuterated nitropicoline intermediate (20) by analogy to a procedure described in CN 104387384. Subsequent sequential treatment with pyrrolidine, and appropriately deuterated N,N-dimethylformamide dimethyl acetal (DMFDMA) intermediate (21) at elevated temperature produces appropriately deuterated pyrrolidinylethenyl intermediate (22). Raney nickel catalyzed reduction of nitro moiety in (22) using hydrazine hydrate, followed by ring closure furnishes appropriately deuterated bromoazaindole (23), and by analogy to a procedure described in WO 2003064413, (23) is treated with sodium methoxide in the presence of copper bromide to furnish appropriately deuterated methoxyazaindole intermediate (24). Finally, hydrolysis of methoxy moiety in (24) with a Lewis acid such as boron tribromide produces appropriately deuterated hydroxyazaindole intermediate (7).

Certain intermediate (14) are commercially available or may be prepared according to published methods: 2-Bromopyridine-d₄ (98 atom %D) (14a); Pyridine-2-d, 6-bromo- (14b) may be prepared according to a procedure described by Alexakis, E. et al., Tetrahedron Letters, 47(29), 5025-5028; 2006 by rhodium catalyzed isotopic exchange of commercially available 2-bromopyridine with deuterium gas.

Intermediate (16) is commercially available: Iodomethane-d₃ (99.5 atom % D) (16a).

Additionally, intermediate (21a), 1,1-dimethoxy-N,N-dimethyl-methan-d-amine, may be prepared by analogy to a procedure described by Pan, Y. et al., Jingxi Huagong Zhongjianti (2008), 38(5), 25-26, 29, from N,N-Dimethylformamide-d₁ (99 atom %D).

Use of appropriately deuterated reagents allows deuterium incorporation at the Y¹³, Y¹⁴, Y¹⁵, Y¹⁶ positions of a compound of Formula I or any appropriate intermediate herein, e.g., about 90%, about 95%, about 97%, or about 99% deuterium incorporation at any Y¹³, Y¹⁴, Y¹⁵, and/or Y¹⁶.

Appropriately deuterated intermediate (2), for use in the preparation of compounds of Formula I according to Scheme 1, may be prepared from corresponding deuterated reagents as exemplified in Scheme 5.

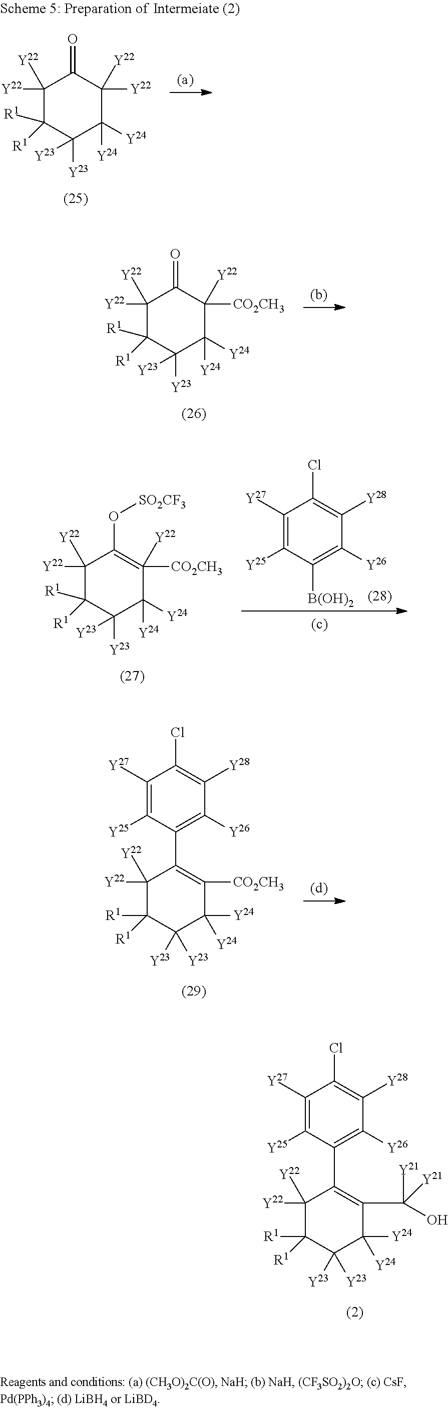

In a manner analogous to a procedure described by Giedt, R. et al., Bioconjugate Chemistry, 25(11), 2081-2085; 2014, appropriately deuterated alkylketone intermediate (25) is treated with dimethylcarbonate in the presence of a base such as sodium hydride to furnish appropriately deuterated ketoester intermediate (26) which is subsequently treated with triflic anhyride in the presence of sodium hydride affording appropriately deuterated triflate intermediate (27). Palladium catalyzed Suzuki cross-coupling of (27) with appropriately deuterated boronic acid intermediate (28) using cesium fluoride as base, or using other conditions known to those skilled in the art provides appropriately deuterated tetrahydrobiphenyl carboxylate intermediate (29). Reduction of the ester moiety in (29) with a reducing agent such as lithium borohydride or lithium borodeuteride furnishes correspondingly and appropriately deuterated alcohol intermediate (2).

Use of appropriately deuterated reagents allows deuterium incorporation at the Y²¹, Y²², Y²³, Y²⁴, Y²⁵, Y²⁶, Y²⁷, Y²⁸, R¹ positions of a compound of Formula I or any appropriate intermediate herein, e.g., about 90%, about 95%, about 97%, or about 99% deuterium incorporation at any Y²¹, Y²², Y²³, Y²⁴, Y²⁵, Y²⁶, Y²⁷, Y²⁸, and/or R¹.

Appropriately deuterated intermediate (25), for use in the preparation of intermediate (2) according to Scheme 5, which is used to prepare compounds of Formula I according to Scheme 1, may be prepared from corresponding deuterated reagents exemplified in Scheme 6.

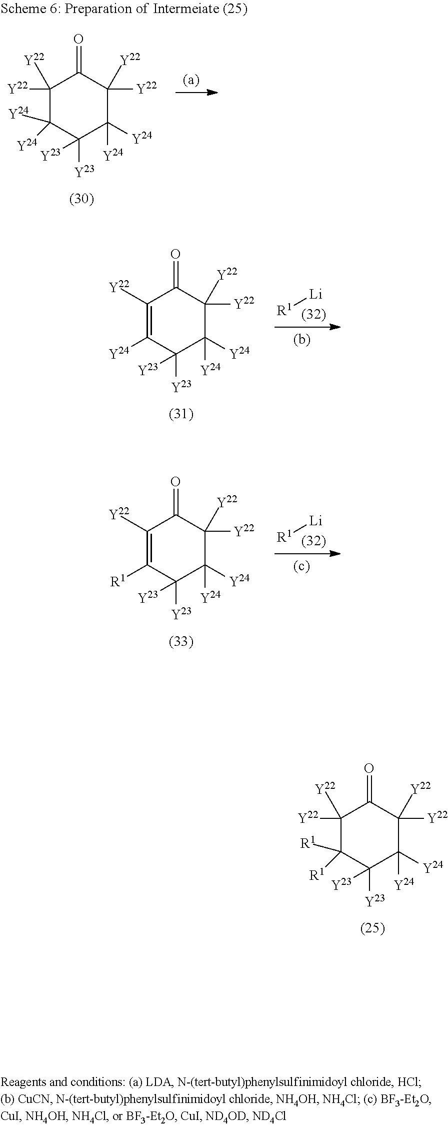

By analogy to a procedure described by Mukaiyama, T. et al., Chemistry Letters, (11), 1250-1252; 2000, dehydrogenation of appropriately deuterated ketone intermediate (30) using N-(tert-butyl)phenylsulfinimidoyl chloride in the presence of a lithium amide base such as lithium diisopropylamine (LDA) furnishes appropriately deuterated enone intermediate (31). In a manner analogous to a procedure described by Matsuo, J. et al., Chemical Communications (Cambridge, United Kingdom), (18), 2399-2401; 2005, or by Kerr, W. et al., Organic & Biomolecular Chemistry, 4(1), 47-50; 2006, in situ treatment of appropriately deuterated methyl lithium intermediate (32) with copper cyanide affords appropriately deuterated higher-order dialkyl cyanocuprate, and subsequent conjugate addition to (31), followed by dehydrogenation with excess N-(tert-butyl)phenylsulfinimidoyl chloride furnishes appropriately deuterated alkyl enone intermediate (33). Finally, in situ treatment of appropriately deuterated methyl lithium intermediate (32) with copper iodide affords appropriately deuterated lower-order dialkylcuprate, and conjugate addition to (33) in the presence of boron trifluoride etherate (BF₃.Et₂O) produces appropriately deuterated dialkylketone intermediate (25), by analogy to a procedure described by Lipshutz, B. et al., Journal of the American Chemical Society, 111(4), 1351-8; 1989.

Certain intermediates (30) are commercially available: Cyclohexanone-2,2,3,3,4,4,5,5,6,6-d₁₀ (98 atom %D) (30a), Cyclohexanone-2,2,6,6-d₄ (98 atom %D) (30b); Cyclohexanone-3,3,4,4,5,5-d₆ (98 atom %D) (30c). The following intermediates (30) may be prepared according to published methods: Cyclohexanone-2,2,4,4,6,6-d₆ (30d) and Cyclohexanone-4,4-d₂ (30e) may be prepared according to a procedure described in WO 2012151343; Cyclohexanone-2,2,3,3,5,5,6,6-d₈ (30f) and Cyclohexanone-3,3,5,5-d₄ (30g) may be prepared according to a procedure described by Lompa-Krzywien, L. et al., Journal of Labelled Compounds (1973), 9(2), 331-8; Cyclohexanone-2,2,3,3,4,4-d₆ (30h) may be prepared according to a procedure described in EP2566869; and Cyclohexanone-2,2,3,3,6,6-d₆ (30i) may be prepared according to a procedure described by Stibbe, W. et al., Journal of Labelled Compounds and Radiopharmaceuticals (1979), 16(4), 567-77.

Methyl-d₃-lithium (99 atom %D) (32a) is commercially available.

Use of appropriately deuterated reagents allows deuterium incorporation at the Y²², Y²³, Y²⁴, R¹ positions of a compound of Formula I or any appropriate intermediate herein, e.g., about 90%, about 95%, about 97%, or about 99% deuterium incorporation at any Y²², Y²³, Y²⁴, and/or R¹.

Appropriately deuterated intermediate (28), for use in the preparation of intermediate (2) according to Scheme 5, which is used to prepare compounds of Formula I according to Scheme 1, may be prepared from corresponding deuterated reagents exemplified in Scheme 7.

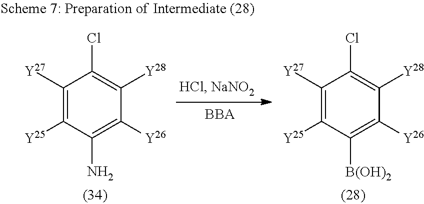

In a manner analogous to a procedure described by Zhao, C. et al., Synlett, 25(11), 1577-1584, 2014, diazotization of appropriately deuterated aniline intermediate (34) followed by treatment with bis-boric acid (BBA) produces appropriately deuterated boronic acid intermediate (28).

The following intermediates (34) are commercially available: 4-Chloroaniline-2,3,5,6-d₄ (98 atom % D) (34a); and 4-Chloroaniline-2,6-d₂ (98 atom %D) (34b). Benzen-3,5-d₂-amine, 4-chloro- (34c) may prepared in accordance with a procedure described by Suehiro, T. et al., Bulletin of the Chemical Society of Japan, 60(9), 3321-30; 1987.

Use of appropriately deuterated reagents allows deuterium incorporation at the Y²⁵, Y²⁶, Y²⁷, Y²⁸ positions of a compound of Formula I or any appropriate intermediate herein, e.g., about 90%, about 95%, about 97%, or about 99% deuterium incorporation at any Y²⁵, Y²⁶, Y²⁷, and/or Y²⁸.

Appropriately deuterated intermediate (5), for use in the preparation of compounds of Formula I according to Scheme 1, may be prepared from corresponding deuterated reagents exemplified in Scheme 8.

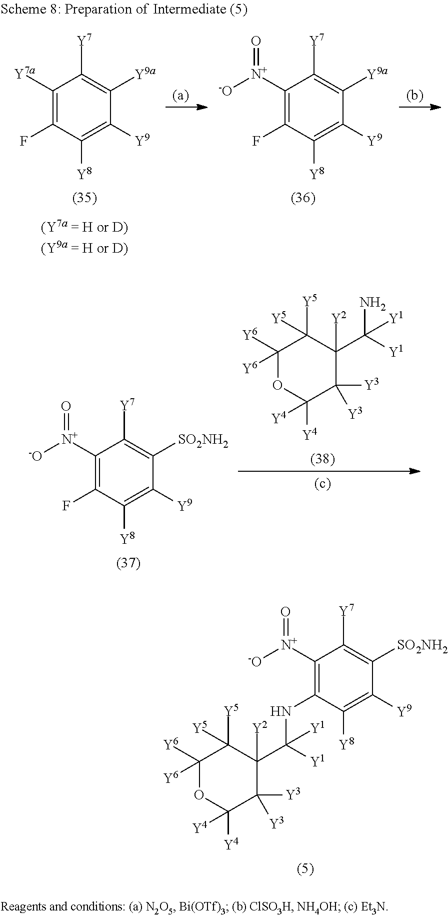

In a manner analogous to a procedure described by Qian, H. et al., Letters in Organic Chemistry, 11(7), 509-512; 2014, selective nitration of appropriately deuterated aryl fluoride intermediate (35) using nitrogen oxide in the presence of bismuth triflate furnishes appropriately deuterated nitrobenzene intermediate (36). Subsequent sulfonylation with chlorosulfonic acid followed by treatment with ammonium hydroxide, by analogy to a procedure described in US20020055631, affords appropriately deuterated sulfonamide intermediate (37). Finally, by analogy to a procedure described in WO2012058392, nucleophilic displacement of fluoride moiety in appropriately deuterated aryl sulfonamide intermediate (37) with appropriately deuterated pyranomethylamine (38) in the presence of triethylamine produces appropriately deuterated sulfonamide intermediate (5).

Fluorobenzene-d₅ (98 atom %D) (35a) is commercially available.

Use of appropriately deuterated reagents allows deuterium incorporation at the Y¹, Y², Y³, Y⁴, Y⁵, Y⁶, Y⁷, Y⁸, Y⁹ positions of a compound of Formula I or any appropriate intermediate herein, e.g., about 90%, about 95%, about 97%, or about 99% deuterium incorporation at any Y¹, Y², Y³, Y⁴, Y⁵, Y⁶, Y⁷, Y⁸, and/or Y⁹.

Appropriately deuterated intermediate (38), for use in the preparation of intermediate (5) according to Scheme 8, which is used to prepare compounds of Formula I according to Scheme 1, may be prepared from corresponding deuterated reagents exemplified in Scheme 9.

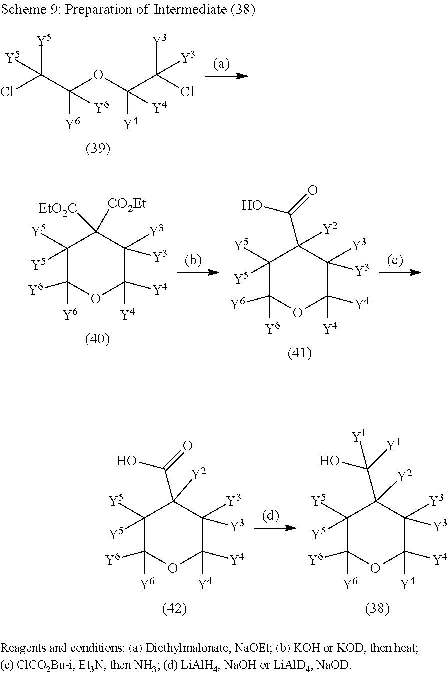

In a manner analogous to a procedure described in WO 2009144473, alkylation of diethylmalonate with appropriately deuterated chloroethyl ether intermediate (39) in the presence of a base such as sodium ethoxide furnishes appropriately deuterated tetrahydropyran dicarboxylate intermediate (40). Hydrolysis of dicarboxylate moiety in (40) using a base such as KOH or KOD, followed by decarboxylation at elevated temperature affords correspondingly and appropriately deuterated tetrahydropyran carboxylic acid intermediate (41). Subsequent activation with isobutyl chloroformate, followed by treatment with ammonia affords appropriately deuterated amide intermediate (42), by analogy to a procedure described in WO 2015177367 or by common methods known in the art. Finally, in a manner analogous to a procedure described in WO 2005066126, reduction of amide in (42) using lithium aluminium hydride or lithium aluminium deuteride produces correspondingly and appropriately deuterated pyranomethylamine (38).

Certain intermediate (39) may be prepared according to published procedure: Ethane-1,1-d₂, 2,2′-oxybis[1-chloro-(39a) may be prepared according to a procedure described by Buchanan, G. et al., Canadian Journal of Chemistry (1993), 71(7), 951-9. Ethane-1,1-d₂, 2-chloro-1-(2-chloroethoxy)-(39b) and Ethane-1,1-d₂, 1-chloro-2-(2-chloroethoxy)-(39c) may be produced according to a procedure described by Lown, J. et al., Journal of Organic Chemistry (1982), 47(11), 2027-2033. Bis(2-chloroethyl)-d₈ Ether (98 atom % D) (39d) is commercially available.

Use of appropriately deuterated reagents allows deuterium incorporation at the Y¹, Y², Y³, Y⁴, Y⁵, Y⁶ positions of a compound of Formula I or any appropriate intermediate herein, e.g., about 90%, about 95%, about 97%, or about 99% deuterium incorporation at any Y¹, Y², Y³, Y⁴, Y⁵ and/or Y⁶.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Pharmaceutical Compositions

The invention also provides pharmaceutical compositions comprising an effective amount of a compound of Formula I:

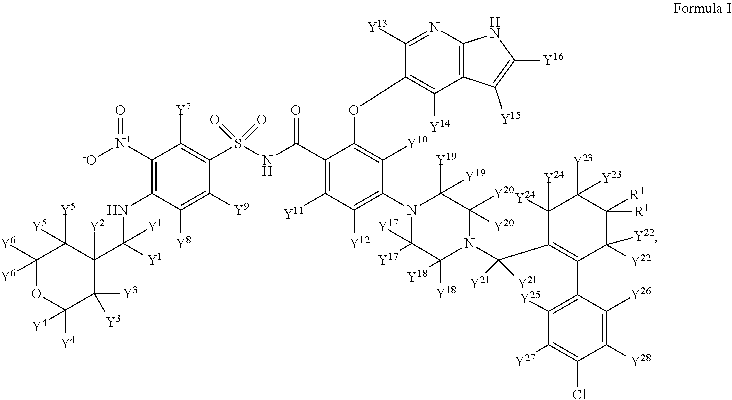

or a pharmaceutically acceptable salt thereof, wherein each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium; each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and each R¹ is independently selected from —CH₃, —CH₂D, —CHD₂ and —CD₃; wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises deuterium; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants, excipients, and vehicles that may be used in the pharmaceutical compositions of this invention include, but are not limited to, ion exchangers, alumina, aluminum stearate, lecithin, serum proteins, such as human serum albumin, buffer substances such as phosphates (e.g., phosphate-buffered saline, etc.), glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni AC, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures of any of the foregoing. The coatings may optionally be further covered by a suitable topcoat of fluorosilicone, polysaccharides, polyethylene glycol, phospholipids or combinations of any of the foregoing to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises one or more additional therapeutic agents. The additional therapeutic agent(s) may be selected from any compound or therapeutic agent known to have or that demonstrates advantageous properties when administered with a compound having the same mechanism of action as venetoclax. Such agents include those indicated as being useful in combination with venetoclax, including but not limited to, those described in US 2016/113925, US 2015/320755, WO 2015/130585, WO 2015/007714, US 2014/248262, and US 2015/174138.

Preferably, the additional therapeutic agent is an agent useful in the treatment of a disease or condition selected from chronic lymphocytic leukemia, acute myeloid leukemia, diffuse large B -cell leukemia, follicular lymphoma, mantle cell lymphoma, non-Hodgkin's lymphoma, multiple myeloma, Waldenstrom's macroglobulinemia, myelodysplasia, amyloidosis, and systemic lupus erythematosus.

In one embodiment, the additional therapeutic agent is selected from cobimetinib, idasanutlin, rituximab, bendamustine, obinutuzumab, duvelisib, ibrutinib, polatuzumab vedotin, rifampicin, azacitidine, decitabine, chlorambucil, bortezomib, and dexamethasone, or any combination of the foregoing.

In another embodiment, the invention provides separate dosage forms of a compound of this invention and one or more of any of the above-described additional therapeutic agents, wherein the compound and additional therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from about 5 mg to about 2000 mg per day, from about 10 mg to about 1200 mg per day, from about 20 mg to about 800 mg per day, from about 50 mg to about 600 mg per day, from about 100 mg to about 400 mg per day, or from about 200 mg to about 300 mg per day.

In one embodiment, an effective amount of a compound of this invention can range from about 0.1 mg/kg to about 100 mg/kg body weight, from about 1 mg/kg to about 50 mg/kg, or from about 10 mg/kg to about 20 mg/kg.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for venetoclax.

For pharmaceutical compositions that comprise one or more additional therapeutic agents, an effective amount of the additional therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

Some of the additional therapeutic agents referenced above may act synergistically with the compounds of this invention. When this occurs, it will allow the effective dosage of the additional therapeutic agent and/or the compound of this invention to be reduced from that required in a monotherapy. This has the advantage of minimizing toxic side effects of either the additional therapeutic agent or a compound of this invention, synergistically improving efficacy, improving ease of administration or use and/or reduced overall expense of compound preparation or formulation.

### Methods of Treatment

In another aspect, the invention provides a method of modulating the activity of Bcl-2 in a cell, comprising contacting a cell with a compound of Formula I:

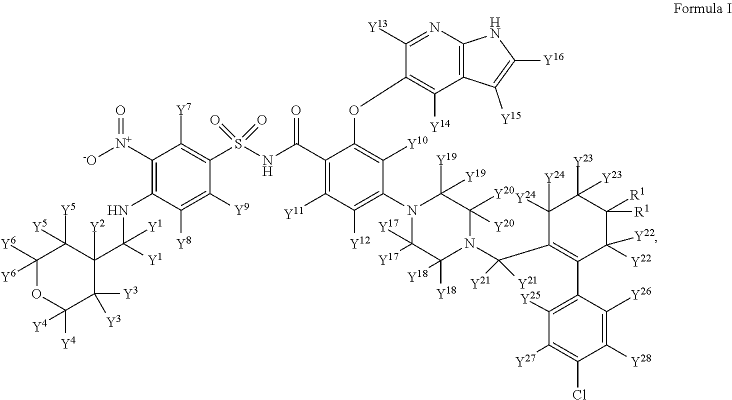

or a pharmaceutically acceptable salt thereof, wherein each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium; each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and each R¹ is independently selected from —CH₃, —CH₂D, —CHD₂ and —CD₃; wherein at least one of Y¹, Y² , Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴ Y²⁵, Y²⁶, Y²⁷, and R¹ comprises deuterium. Some embodiments provide a method of inhibiting the anti-apoptotic activity of Bcl-2 in a cell, comprising contacting the cell with a compound or a pharmaceutical composition of the present invention. In some embodiments, the cell is contacted in vitro. In some embodiments, the cell is contacted in vivo. In some embodiments, the cell is contacted ex vivo.

According to another aspect, the invention provides a method of treating a disease that is beneficially treated by a compound of Formula I:

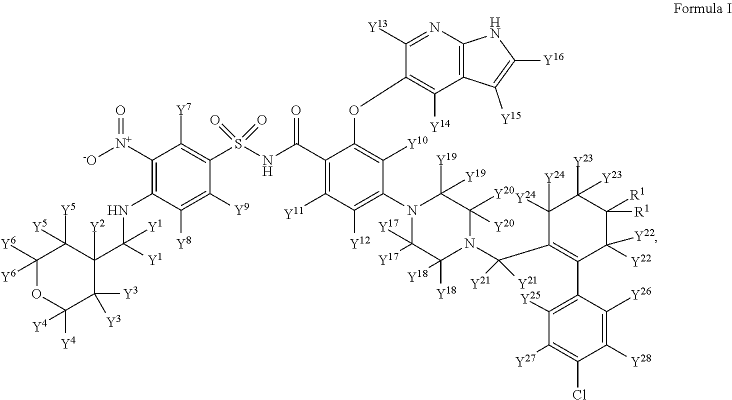

or a pharmaceutically acceptable salt thereof, wherein each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium; each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and each R¹ is independently selected from —CH₃, —CH₂D, —CHD₂ and —CD₃; wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸ and R¹ comprises deuterium, in a subject in need thereof, comprising administering to the subject an effective amount of a compound or a composition of this invention. In one embodiment, the subject is a patient in need of such treatment. In certain embodiments, the subject is a human. Such diseases are well known in the art and are disclosed in, but not limited to the diseases disclosed in the following patents and published applications: WO 2010/138588, US 2011/124628, US 2012108590, WO 2012058392, WO 2012071336, WO 2012071374, WO 2013192274, WO 2014039855, US 2015150870, WO 2015130585, WO 2015160975, and US 2016113925.

Such diseases include, but are not limited to, acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, amyloidosis, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelodysplasia, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenstrom's macroglobulinemia, testicular tumors, uterine cancer, Wilms' tumor, embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer, acquired immunodeficiency disease syndrome (AIDS), autoimmune lymphoproliferative syndrome, hemolytic anemia, inflammatory diseases, and thrombocytopenia, acute or chronic immune disease associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcoholinduced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycaemia, autoimmune neutropaenia, autoimmune thrombocytopaenia, autoimmune thyroid disease, B cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinaemia), conjunctivitis, connective tissue disease associated interstitial lung disease, contact dermatitis, Coombs positive haemolytic anaemia, cor pulmonale, Creutzfeldt-Jakob disease, cryptogenic autoimmune hepatitis, cryptogenic fibrosing alveolitis, culture negative sepsis, cystic fibrosis, cytokine therapy associated disorders, Crohn's disease, dementia pugilistica, demyelinating diseases, dengue hemorrhagic fever, dermatitis, dermatitis scleroderma, dermatologic conditions, dermatomyositis/polymyositis associated lung disease, diabetes, diabetic arteriosclerotic disease, diabetes mellitus, Diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine, receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram negative sepsis, gram positive sepsis, granulomas due to intracellular organisms, group B streptococci (GBS) infection, Grave's disease, haemosiderosis associated lung disease, hairy cell leukemia, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpurea, Hepatitis A, Hepatitis B, Hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopaenia, idiopathic pulmonary fibrosis, idiopathic thrombocytopaenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anaemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease associated lung disease, monoclonal gammopathy, multiple myeloma, multiple systems degenerations (Mencel Dejerine-Thomas Shi-Drager and Machado-Joseph), myalgic encephalitis/Royal Free Disease, myasthenia gravis, microscopic vasculitis of the kidneys, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, Non-alcoholic Steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, Progressive supranucleo Palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis associated interstitial lung disease, rheumatoid spondylitis, sarcoidosis, Schmidt's syndrome, scleroderma, senile chorea, Senile Dementia of Lewy body type, sepsis syndrome, septic shock, seronegative arthropathies, shock, sickle cell anemia, Sjogren's disease associated lung disease, Sjorgren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, spondyloarthopathy, sporadic, polyglandular deficiency type I sporadic, polyglandular deficiency type II, Still's disease, streptococcal myositis, stroke, structural lesions of the cerebellum, Subacute sclerosing panencephalitis, sympathetic ophthalmia, Syncope, syphilis of the cardiovascular system, systemic anaphylaxis, systemic inflammatory response syndrome, systemic onsetjuvenile rheumatoid arthritis, systemic lupus erythematosus, systemic lupus erythematosus-associated lung disease, systemic sclerosis, systemic sclerosis-associated interstitial lung disease, T-cell or FAB ALL, Takayasu's disease/arteritis, telangiectasia, Th2 Type and Th1 Type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy.

In one particular embodiment, the method of this invention is used to treat a disease or condition selected from chronic lymphocytic leukemia, acute myeloid leukemia, diffuse large B-cell leukemia, follicular lymphoma, myelodysplasia, amyloidosis, mantle cell lymphoma, non-Hodgkin's lymphoma, multiple myeloma, Waldenstrom's macroglobulinemia, and systemic lupus erythematosus in a subject in need thereof.

In another particular embodiment, the method of this invention is used to treat chronic lymphocytic leukemia in a subject in need thereof.

Identifying a subject in need of such treatment can be in the judgment of a subject or a health care professional and can be subjective (e.g., opinion) or objective (e.g., measurable by a test or diagnostic method).

In another embodiment, any of the above methods of treatment comprises the further step of co-administering to the subject in need thereof one or more additional therapeutic agents. The choice of additional therapeutic agent may be made from any additional therapeutic agent known to be useful for co-administration with venetoclax. The choice of additional therapeutic agent is also dependent upon the particular disease or condition to be treated. Examples of additional therapeutic agents that may be employed in the methods of this invention are those set forth above for use in combination compositions comprising a compound of this invention and an additional therapeutic agent.

In particular, the combination therapies of this invention include co-administering a compound of Formula I and one or more additional therapeutic agents to a subject in need thereof for treatment of the following conditions (with the particular additional therapeutic agent indicated in parentheses following the indication): acute myeloid leukemia (cobimetinib and idasanutlin), chronic lymphocytic leukemia (rituximab; rituximab and bendamustine; bendamustine and obinutuzumab; duvelisib; obinutuzumab; ibrutinib; ibrutinib and obinutuzumab; obinutuzumab and chlorambucil), non-Hodgkin's lymphoma/diffuse large B-cell lymphoma (bendamustine and rituximab), follicular lymphoma/diffuse large B-cell lymphoma (obinutuzumab and polatuzumab), follicular lymphoma (rituximab; rituximab and bendamustine), diffuse large B-cell lymphoma (ibrutinib and rituximab), mantle cell lymphoma (ibrutinib; ibrutinib and obinutzumab), non-Hodgkin's lymphoma (rifampicin), acute myelogenous leukemia (azacitidine and decitabine), myelodysplasia (azacitidine), amyloidosis (dexamethasone), and multiple myeloma (dexamethasone and bortezomib; dexamethasone and carfilzomib).

The term “co-administered” as used herein means that the additional therapeutic agent may be administered together with a compound of this invention as part of a single dosage form (such as a composition of this invention comprising a compound of the invention and an additional therapeutic agent as described above) or as separate, multiple dosage forms. Alternatively, the additional agent may be administered prior to, consecutively with, or following the administration of a compound of this invention. In such combination therapy treatment, both the compounds of this invention and the additional therapeutic agent(s) are administered by conventional methods. The administration of a composition of this invention, comprising both a compound of the invention and an additional therapeutic agent to a subject does not preclude the separate administration of that same therapeutic agent or any other additional therapeutic agent or any compound of this invention to said subject at another time during a course of treatment.

Effective amounts of these additional therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the additional therapeutic agent's optimal effective-amount range.

In one embodiment of the invention, where an additional therapeutic agent is administered to a subject, the effective amount of the compound of this invention is less than its effective amount would be where the additional therapeutic agent is not administered. In another embodiment, the effective amount of the additional therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described additional therapeutic agents in the manufacture of a medicament, either as a single composition or as separate dosage forms, for treatment in a subject of a disease, disorder or symptom set forth above. Another aspect of the invention is a compound of Formula I for use in the treatment in a subject of a disease, disorder or symptom thereof delineated herein.

### Example 1. Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. The relevant teachings of all patents, published applications and references cited herein are incorporated by reference in their entirety.

1. A compound of Formula I: 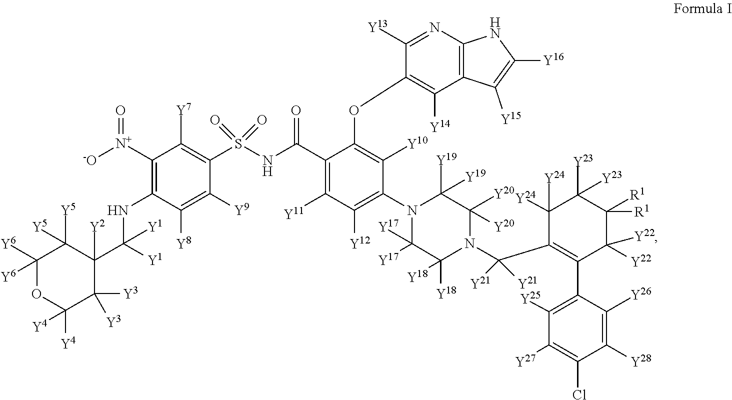
or a pharmaceutically acceptable salt thereof, wherein:
each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium;
each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and
each R¹ is independently selected from —CH₃, —CH₂D, —CHD₂ and —CD₃;
wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, and Y²⁸, and R¹ comprises deuterium;
provided that when each of Y¹⁷, Y¹⁸, Y¹⁹, and Y²⁰ is deuterium, then at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y²¹, Y²², Y²³, Y²⁴, Y²⁵, Y²⁶, Y²⁷, Y²⁸, and R¹ comprises deuterium.

2. The compound of claim 1, wherein each R¹ is the same, each Y¹ is the same, each Y³ is the same, each Y⁴ is the same, each Y⁵ is the same, each Y⁶ is the same, each Y¹⁷ is the same, each Y¹⁸ is the same, each Y¹⁹ is the same, each Y²⁰ is the same, each Y²¹ is the same, each Y²² is the same, each Y²³ is the same, and each Y²⁴ is the same.

3. The compound of claim 2, wherein R¹ is —CH₃ or —CD₃.

4. The compound of claim 1, wherein Y² and each Y¹, Y³, Y⁴, Y⁵, and Y⁶ are deuterium.

5. The compound of claim 1, wherein each Y¹, Y⁴, and Y⁶ is deuterium, and Y² and each Y³ and Y⁵ are hydrogen.

6. The compound of claim 1, wherein each of Y¹³, Y¹⁴, and Y¹⁶ is deuterium.

7. The compound of claim 1, wherein each Y¹⁷ and Y¹⁹ is deuterium, and each Y¹⁸ and Y²⁰ is hydrogen.

8. The compound of claim 1, wherein each Y¹⁷ and Y¹⁹ is hydrogen, and each Y¹⁸ and Y²⁰ is deuterium.

9. The compound of claim 1, wherein each Y²¹, Y²², Y²³, and Y²⁴ is deuterium.

10. The compound of claim 1, wherein each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is hydrogen; Y² and each Y¹, Y³, Y⁴, Y⁵, and Y⁶ are the same; each Y¹⁷ and Y¹⁹ is the same; each Y¹⁸ and Y²⁰ is the same; each Y²¹, Y²², Y²³, and Y²⁴ is the same; each R¹ is the same; and the compound is selected from any one of the compounds set forth in Table 1 below:
TABLE 1

| Compound | Y¹⁻⁶ | Y¹⁷/Y¹⁹ | Y¹⁸/Y²⁰ | Y²¹⁻²⁴ | R¹ |
|---|---|---|---|---|---|
| 101 | D | H | H | H | CH₃ |
| 102 | D | D | H | H | CH₃ |
| 103 | D | H | D | H | CH₃ |
| 104 | D | H | H | D | CH₃ |
| 105 | D | D | D | H | CH₃ |
| 106 | D | H | D | D | CH₃ |
| 107 | D | D | H | D | CH₃ |
| 108 | D | D | D | D | CH₃ |
| 109 | D | H | H | H | CD₃ |
| 110 | D | D | H | H | CD₃ |
| 111 | D | H | D | H | CD₃ |
| 112 | D | H | H | D | CD₃ |
| 113 | D | D | D | H | CD₃ |
| 114 | D | H | D | D | CD₃ |
| 115 | D | D | H | D | CD₃ |
| 116 | D | D | D | D | CD₃ |
| 117 | H | D | H | H | CH₃ |
| 118 | H | H | D | H | CH₃ |
| 119 | H | H | H | D | CH₃ |
| 120 | H | D | D | H | CH₃ |
| 121 | H | H | D | D | CH₃ |
| 122 | H | D | H | D | CH₃ |
| 123 | H | D | D | D | CH₃ |
| 124 | H | H | H | H | CD₃ |
| 125 | H | D | H | H | CD₃ |
| 126 | H | H | D | H | CD₃ |
| 127 | H | H | H | D | CD₃ |
| 128 | H | D | D | H | CD₃ |
| 129 | H | H | D | D | CD₃ |
| 130 | H | D | H | D | CD₃ |
| 131 | H | D | D | D | CD₃, |

or a pharmaceutically acceptable salt of any of the foregoing, wherein any atom not designated as deuterium is present at its natural isotopic abundance.

11. The compound of claim 1, wherein each of Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is hydrogen; each Y¹, Y⁴, and Y⁶ is deuterium; Y² and each Y³ and Y⁵ are hydrogen; each Y¹⁷ and Y¹⁹ is the same; each Y¹⁸ and Y²⁰ is the same; each Y²¹, Y²², Y²³, and Y²⁴ is the same; each R¹ is the same; and the compound is selected from any one of the compounds set forth in Table 2 below:
TABLE 2

| Compound | Y¹⁷/Y¹⁹ | Y¹⁸/Y²⁰ | Y²¹⁻²⁴ | R¹ |
|---|---|---|---|---|
| 201 | H | H | H | CH₃ |
| 202 | D | H | H | CH₃ |
| 203 | H | D | H | CH₃ |
| 204 | H | H | D | CH₃ |
| 205 | D | D | H | CH₃ |
| 206 | H | D | D | CH₃ |
| 207 | D | H | D | CH₃ |
| 208 | D | D | D | CH₃ |
| 209 | H | H | H | CD₃ |
| 210 | D | H | H | CD₃ |
| 211 | H | D | H | CD₃ |
| 212 | H | H | D | CD₃ |
| 213 | D | D | H | CD₃ |
| 214 | H | D | D | CD₃ |
| 215 | D | H | D | CD₃ |
| 216 | D | D | D | CD₃, |

or a pharmaceutically acceptable salt of any of the foregoing, wherein any atom not designated as deuterium is present at its natural isotopic abundance.

12. The compound of any one of claim 1, wherein any atom not designated as deuterium is present at its natural isotopic abundance.

13. The compound of claim 1, wherein the deuterium incorporation at each designated deuterium atom is at least 90%.

14. The compound of claim 1, wherein the deuterium incorporation at each designated deuterium atom is at least 95%.

15. The compound of claim 1, wherein the deuterium incorporation at each designated deuterium atom is at least 97%.

16. A pharmaceutical composition comprising a compound of Formula I: 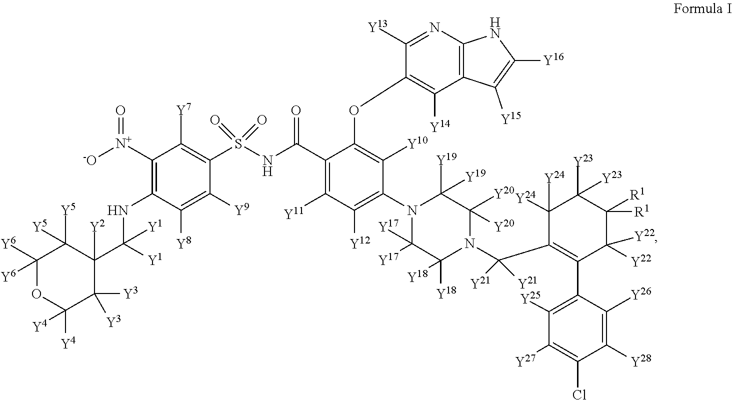
or a pharmaceutically acceptable salt thereof, wherein:
each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium;
each of Y², Y⁷, Y⁸, Y⁹, Y¹⁰, Y¹¹, Y¹², Y¹³, Y¹⁴, Y¹⁵, Y¹⁶, Y²⁵, Y²⁶, Y²⁷, and Y²⁸ is independently hydrogen or deuterium; and
each R¹ is independently selected from —CH₃, —CH₂D, —CHD₂ and —CD₃; wherein at least one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises deuterium; and a pharmaceutically acceptable carrier.

17. The pharmaceutical composition of claim 16, further comprising an additional therapeutic agent selected from cobimetinib, idasanutlin, rituximab, bendamustine, obinutuzumab, duvelisib, ibrutinib, polatuzumab vedotin, rifampicin, azacitidine, decitabine, chlorambucil, bortezomib, or dexamethasone, or any combination of the foregoing.

18. A method of inhibiting the anti-apoptotic activity of B-cell lymphoma (Bc1-2) protein in a cell, comprising contacting the cell with a compound of Formula I: 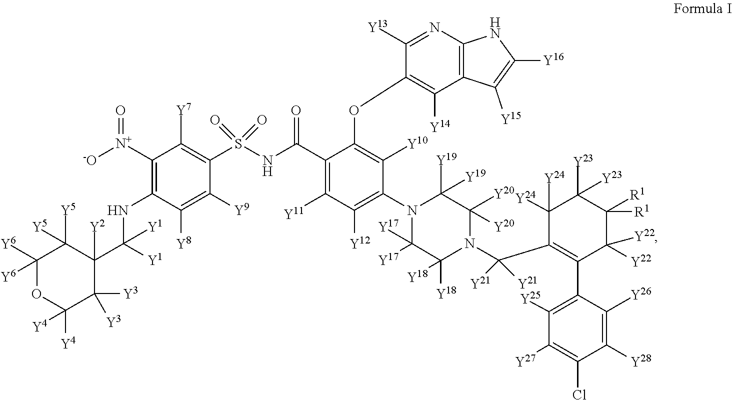
or a pharmaceutically acceptable salt thereof, wherein:
each Y¹, Y³, Y⁴, Y⁵, Y⁶, Y¹⁷, Y¹⁸, Y¹⁹, Y²⁰, Y²¹, Y²², Y²³, and Y²⁴ is independently hydrogen or deuterium;
each of Y², Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, Y²⁴, Y²⁵, Y²⁶, Y²⁷, Y²⁸, and R¹ comprises deuterium;
or a composition comprising the compound of Formula I, or a pharmaceutically acceptable salt thereof, and a pharmaceutically acceptable carrier.

19. A method of treating a disease selected from the group consisting of chronic lymphocytic leukemia, acute myeloid leukemia, diffuse large B-cell leukemia, follicular lymphoma, mantle cell lymphoma, non-Hodgkin's lymphoma, multiple myeloma, Waldenstrom's macroglobulinemia, and systemic lupus erythematosus, comprising administering to a subject in need thereof a composition of claim 16.

20. The method of claim 19, wherein the composition is administered with an additional therapeutic agent selected from cobimetinib, idasanutlin, rituximab, bendamustine, obinutuzumab, duvelisib, ibrutinib, polatuzumab vedotin, rifampicin, azacitidine, decitabine, chlorambucil, bortezomib, or dexamethasone, or any combination of the foregoing.
